(19) United States
(12) Patent Application Publication
(10) Pub. No.: US 2023/0278078 A1
(43) Pub. Date: Sep. 7, 2023

(54) Medical Imaging Apparatus with a Functional Housing Shell

(71) Applicant: Siemens Healthcare GmbH, Erlangen (DE)

(72) Inventors: Michael Ott, Speinshart (DE); Martin Seifert, Bayreuth (DE); Erik Weisbrod, München (DE); Stefan Stegmeier, München (DE)

(73) Assignee: Siemens Healthcare GmbH, Erlangen (DE)

(21) Appl. No.: 18/115,900

(22) Filed: Mar. 1, 2023

(30) Foreign Application Priority Data
Mar. 2, 2022 (EP) 22159740.4

(51) Int. Cl. B08B 7/00; B08B 3/08; A61L 2/28; A61L 2/04; A61L 2/08; A61L 2/20; A61L 2/18

(52) U.S. Cl. CPC B08B 7/0057; B08B 3/08; B08B 7/0071; A61L 2/28; A61L 2/04; A61L 2/08; A61L 2/202; A61L 2/183; A61L 2/208; A61L 2/186; A61L 2202/24; A61L 2202/17; A61L 2202/14

(57) ABSTRACT

The disclosure is based on a medical imaging apparatus with a scanner, a patient support apparatus and a housing, which is arranged on the scanner and/or on the patient support apparatus, wherein the housing comprises at least one functional housing shell, wherein the at least one functional housing shell comprises at least one sensor element for detection of a hygiene status on the at least one functional housing shell, at least one output element, which is embodied for a visual display of the hygiene status, and at least one cleaning element for cleaning a surface of the at least one functional housing shell.

### CROSS REFERENCE TO RELATED APPLICATIONS

This patent application claims priority to European Patent Application No. 22159740.4, filed Mar. 2, 2022, which is incorporated herein by reference in its entirety.

### BACKGROUND

### Field

The present disclosure relates to a medical imaging apparatus with a scanner unit, a patient support apparatus and a housing unit, which is arranged on the scanner unit and/or on the patient support apparatus, wherein the housing unit comprises at least one functional housing shell. Furthermore, the disclosure is based on a method for cleaning a surface of a functional housing shell of a medical imaging apparatus.

### Related Art

A medical imaging examination, such as for example a magnetic resonance examination, a computed tomography examination (CT examination) etc., and/or a preparation for a medical imaging examination can result in a plurality of touches and/or contacts by a person, especially the patient and/or the medical operating personnel carrying out the medical imaging examination, on a surface of the medical imaging apparatus. Moreover, during a medical imaging examination there can also be further contaminations of surfaces of the medical imaging apparatus, such as for example coughing on a surface etc. After the medical imaging examination these surfaces contaminated and/or made dirty by the patient and/or the medical operating personnel must be cleaned before another medical imaging examination on a further patient can be carried out. Previously a medical operator and/or a cleaner had to decide for themselves after a medical imaging examination which of the surfaces of the medical imaging apparatus was to be cleaned and how such cleaning would be undertaken.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 schematically shows a medical imaging apparatus with a functional housing shell according to an exemplary embodiment of the present disclosure.

FIG. 2 shows a functional housing shell, in a cross-sectional diagram, according to an exemplary embodiment of the present disclosure.

FIG. 3 shows a functional housing shell, in a cross-sectional diagram, according to an exemplary embodiment of the present disclosure.

FIG. 4 shows a functional housing shell, in a cross-sectional diagram, according to an exemplary embodiment of the present disclosure.

FIG. 5 shows a functional housing shell, in an overhead view, according to an exemplary embodiment of the present disclosure.

FIG. 6 shows the functional housing shell of FIG. 5 in a cross-sectional diagram.

FIG. 7 shows a flowchart of a method for cleaning a surface of a functional housing shell of a medical imaging apparatus, according to an exemplary embodiment of the present disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the present disclosure is to support cleaning of a surface of a medical imaging apparatus.

The disclosure is based on a medical imaging apparatus with a scanner, a patient support apparatus and a housing, which is arranged on the scanner and/or on the patient support apparatus, wherein the housing comprises at least one functional housing shell. In accordance with the disclosure the at least one functional housing shell comprises at least one sensor element for detection of a hygiene status on the at least one functional housing shell, at least one output element, which is embodied for outputting a visual display of the hygiene status, and at least one cleaning element for cleaning a surface of the at least one functional housing shell.

The medical imaging apparatus in this case can for example comprise a magnetic resonance apparatus and/or a computed tomography apparatus and/or a PET apparatus (Positron Emission Tomography apparatus) and/or further medical imaging apparatuses appearing sensible to the person skilled in the art. Accordingly a medical imaging examination can also comprise a magnetic resonance examination and/or a computed tomography examination and/or a PET examination and/or further medical imaging examinations appearing sensible to the person skilled in the art. Moreover, the medical imaging apparatus can also comprise radiation therapy apparatuses.

In an exemplary embodiment, the medical imaging apparatus may include a detector and/or a scanner, which is embodied and/or configured to detect medical imaging data during the medical imaging examination on the patient. If for example the medical imaging apparatus is embodied as a magnetic resonance apparatus, the scanner can comprise a basic magnet for creation of a homogeneous magnetic field, a gradient system for spatial encoding of the detected magnetic resonance data and a radio-frequency antenna unit. The radio-frequency antenna unit in this case can comprise a radio-frequency antenna fixed within the scanner for emitting an excitation pulse. Furthermore, the radio-frequency antenna unit can also comprise a local radio-frequency antenna, which is arranged for detection of magnetic resonance signals around the region of the patient to be examined.

Furthermore, the medical imaging apparatus has a patient support apparatus with a movable patient table. For a medical imaging examination, in particular during preparation for the medical imaging examination, first of all the patient is positioned on the movable patient table of the patient support apparatus. Moreover, accessory units of the medical imaging apparatus required for the impending medical imaging examination are attached to and/or arranged on the patient. Such an accessory unit can for example comprise an EKG unit and/or an infusion unit and/or further units appearing sensible to the person skilled in the art. Moreover, the accessory unit can comprise a support unit, such as for example a support pillow, for correct and/or more comfortable support and/or positioning of the patient on the patient table.

For a medical imaging examination, the patient is subsequently brought into a patient receiving area of the medical imaging apparatus. A Field of View (FOV) and/or an isocenter of the medical imaging apparatus may be arranged with the patient receiving area. The FOV may comprises a detection area of the medical imaging apparatus, within which the conditions for a detection of medical imaging data are present. The isocenter of the medical imaging apparatus may comprises the area and/or point within the medical imaging apparatus having the optimal and/or ideal conditions for the detection of medical imaging data. For example, in a magnetic resonance apparatus, the isocenter comprises the most homogeneous magnet field area.

The housing of the medical imaging apparatus is arranged on the scanner and/or on the patient support apparatus. The housing comprises at least one functional housing shell. In an exemplary embodiment, the at least one functional housing shell is configured to support a detection of a possible contamination of the surface of the functional housing shell and/or a cleaning of the surface the at least one functional housing shell. In an exemplary embodiment, the functional housing shell is arranged on areas of the housing that have a high probability of being contaminated and/or getting dirty during a medical imaging examination. Surfaces of housing shells have a high probability of contamination when they are arranged in an area of contact with the patient and/or the medical operating personnel. For example, surfaces of housing shells of the patient support apparatus and/or of the patient receiving area of the medical imaging apparatus and/or a front area of the medical imaging apparatus are subjected to frequent contact with the patient and/or the medical operating personnel.

The at least one functional housing shell has at least one sensor element for detection of a hygiene status on the at least one functional surface. The hygiene status in this case can comprise a contamination and/or a soiling on the surface of the functional housing shell. Moreover, the hygiene status can also comprise a type of a contamination, such as for example touching or coughing on the surface etc. Furthermore, the hygiene status can also comprise information that a contamination and/or soiling of the surface is present. Moreover, the hygiene status can also comprise information about a cleaning of the surface of the functional housing shell. In an exemplary embodiment, the at least one sensor element is arranged and/or integrated in a layer close to the surface of the at least one functional housing shell for a direct detection.

A contamination and/or a soiling of the surface of the at least one functional housing shell is brought about for example by a direct contact between the patient and/or the medical operating personnel and the surface of the functional housing shell. Moreover, a contamination and/or soiling of the surface of the at least one functional housing shell can also occur by an application of fluids of the patient, for example through coughing or sneezing.

The at least one output element is embodied for a visual display of the hygiene status. To this end the at least one output element may be integrated into the at least one functional housing shell in such a way that the display of the hygiene status is visible directly for a user. For example, an illumination of a red light by the at least one output element can signal to the user that a contamination and/or a soiling of the surface of the functional housing shell is present. On the other hand, an illumination of a green light by the at least one output element can signal to the user that the surface of the functional housing shell is free from contamination and/or soiling. Furthermore, it is also conceivable for an illumination of a blue light by the at least one output element to signal an ongoing cleaning process to the user.

The at least one cleaning element of the at least one functional housing shell is embodied for cleaning the surface of the functional housing shell. In an exemplary embodiment, the at least one cleaning element is arranged and/or integrated here in a layer of the at least one functional housing shell close to the surface. The at least one cleaning element can be embodied in such a way that a cleaning by cleaning personnel is supported, such as for example by provision of a cleaning fluid. Moreover the at least one cleaning element can also be embodied for automatic cleaning of the surface of the at least one functional housing shell.

Moreover, the functional housing shell can also comprise more than one sensor element and/or more than one output element and/or more than one cleaning element.

In an exemplary embodiment, the medical imaging apparatus includes a controller, which is configured to evaluate the data detected by the at least one sensor element and determine a hygiene status for the surface of the at least one functional housing shell. In particular, the at least one output element can also be controlled by the controller with the aid of the hygiene status determined. Furthermore, the controller can also be configured to control the at least one cleaning element in such a way that an automatic cleaning of the surface of the at least one functional housing shell can be undertaken. If the housing has more than one functional housing shell, the controller may be configured to control the multiple shells, such as all functional housing shells.

The disclosure has the advantage that a user, in particular a medical operator and/or a medical cleaner, can advantageously be supported during cleaning of the medical imaging apparatus after a medical imaging examination. In particular in this way a possible contamination of surfaces can be detected automatically and indicated to the user. Visual display enables a user to detect a possible contamination and/or soiling especially quickly and to effectively estimate a possible cleaning effort and/or a cleaning workflow. Moreover, the user can also be given advantageous support during a cleaning of the surfaces because of the at least one cleaning element. This also makes it possible to carry out a cleaning workflow in a time-saving and efficient manner. Moreover, through the visual display, a danger of overlooking a surface during cleaning is also reduced and thus also a danger of infection for a patient is reduced.

In an advantageous development of the inventive medical imaging apparatus there can be provision for the at least one sensor element to comprise a conductivity sensor, a moisture sensor, a temperature sensor, an optical sensor, a chemical and/or electrochemical sensor, a capacitive sensor, a resistive sensor and/or gas sensor. In an exemplary embodiment, in this case a gas sensor based on metal oxide can be employed, which is characterized by a very high sensitivity. In an exemplary embodiment, a flat capacitive sensor is also suitable for detection of contact between a patient and/or the medical operating personnel and the surface of the functional housing shell. Moreover the at least one sensor element can also comprise a combination of a number of, in particular different, sensors so that different parameters for detection of a hygiene status can be detected. In an alternate embodiment of the disclosure further embodiments of the at least one sensor element are also conceivable.

This embodiment of the disclosure makes possible a simple and rapid detection of a contamination and/or soiling. Moreover, in this way a cleaning process can also advantageously be detected. Hence a hygiene workflow can also be adapted to the detected contamination and/or soiling.

In an advantageous development of the inventive medical imaging apparatus there can be provision for the at least one output element to comprise an OLED (organic light-emitting diode), an LED (light-emitting diode), an electrochromic output element and/or an electroluminescent output element. An electroluminescent output element may include a solid-state component, which, as a result of an application of an electrical field and/or of an electrical voltage, sends out and/or emits an electromagnetic radiation, in particular in the form of light. An electrochromic output element may include an output element that can change its optical properties as a result of an application of an electrical field. This embodiment of the disclosure has the advantage that a compact and constructively simple output element can be provided for display of the hygiene status for a user. Moreover, an especially low-cost output element for supporting cleaning of a surface of the functional housing shell can be provided.

In an advantageous development of the inventive medical imaging apparatus there can be provision for the at least one cleaning element to comprise a heating element and/or a fluid channel. In an exemplary embodiment, the heating element comprises a heating wire and/or a heating resistor, which is configured to convert electrical energy into thermal energy. Moreover, the heating element can also comprise a structure made of indium tin oxide (ITO), wherein the structure made of ITO can comprise transparent conductor tracks of for example a heatable transparent layer, in particular a heatable transparent surface layer and/or housing layer. Moreover, the heating element can also have a structure made of zinc aluminum oxide, by means of which a heatable transparent layer, in particular a layer with transparent conductor tracks can be realized. Furthermore, the heating element can also comprise an optically transparent layer made of carbon nanotubes (CNT). Here too a transparent layer with conductor tracks can be realized. For example, a surface and/or an outer housing layer of the at least one functional housing shell can be heated to appr. 200° C. with this. In an exemplary embodiment, the heating takes place within a few seconds through explicit heating pulses.

The fluid channel may include a channel for distribution of a cleaning agent and/or a disinfection agent onto the surface of the at least one functional housing shell. In this case the cleaning agent and/or disinfection agent can comprise a fluid and/or also a gas, such as for example ozone, hydrogen peroxide, chloride, peroxide etc. In an exemplary embodiment, for this purpose the at least cleaning agent has a pump for distribution of the cleaning agent and/or disinfection agent and/or a container for accommodating the cleaning agent and/or disinfection agent. Moreover, the fluid channel can also be embodied for sucking up a cleaning agent that is already distributed onto the surface of the at least one functional surface. This embodiment of the disclosure has the advantage that a simple cleaning of the surface of the at least one functional housing shell can be provided. In particular, in connection with a controller, an automatic cleaning of the surface of the at least one functional housing shell can be provided and thus the load on cleaning personnel can advantageously be relieved and/or said personnel can be supported.

In an alternate embodiment of the disclosure the at least one cleaning element can also comprise a radiation source, which achieves a cleaning and/or decontamination of the surface of the at least one functional housing shell by irradiating the surface. The radiation source can for example comprise a UV-C radiation source. Furthermore, the radiation source can also comprise a HINS (High Intensity Narrow Spectrum) element.

In an advantageous development of the inventive medical imaging apparatus there can be provision for the at least one functional housing shell to have a layer structure with an outer housing layer, wherein the at least one cleaning element and/or the at least one sensor element are arranged in the outer housing layer. The layer structure of the at least one functional housing shell has at least two or more layers. The outer housing layer may have a surface layer, wherein the surface layer comprises that housing layer that is subjected to a contamination and/or soiling. In addition, it can also be that the at least one output element is arranged in the outer housing layer of the functional housing shell. If at least one cleaning element embodied as a heating element is arranged in the outer housing layer, the heating element may be configured such that the heating element is inert on contact with air, such as for example a copper wire and/or a heating element made of carbon.

The layer structure of the at least functional housing shell may have a thickness of 50 μm to 5 mm. In an exemplary embodiment, the layer structure of the at least functional housing shell has a thickness of between 100 μm and 500 μm. In an exemplary embodiment, the layer structure of the at least functional housing shell has a thickness of between 100 μm and 200 μm.

The layer structure can be manufactured by conventional methods, such as for example laminating and/or gluing and/or using a physical vapor deposition (PVD) method and/or a chemical vapor deposition (CVD) method and/or photolithography and/or etching etc. In an exemplary embodiment, the layer structure can also be fabricated by a thermal spray method of metal tracks on plastic and/or 3D printing.

This embodiment of the disclosure has the advantage that a direct detection of a soiling and/or also direct provision of a cleaning of the surface can be achieved. Moreover, components and/or elements of the functional housing shell that require particular protection can be arranged in a further housing layer, such as for example in a housing layer far from the surface.

In an advantageous development of the inventive medical imaging apparatus there can be provision for the layer structure to have at least one further housing layer, in which the at least one output element is arranged. In this way the at least one output element can be arranged protected. If for example the at least one cleaning element comprises a heating element, it can be advantageous for the at least one output element to be arranged in a housing layer which is different from and/or separated from the housing layer comprising the at least one cleaning element.

In an advantageous development of the inventive medical imaging apparatus there can be provision for the outer housing layer to be embodied at least partly transparently. In this context a housing layer embodied at least partly transparently should in particular be understood as the housing layer being embodied at least in part transparently for visible light, in particular for light with a wavelength from 380 nm to 750 nm. In this case the outer housing layer can comprise areas that are embodied transparently and areas that are embodied non-transparently. In an exemplary embodiment, those areas of the outer housing layer that cover an output element of the functional housing shell are embodied transparently. This embodiment of the disclosure has the advantage that at least one output element can be arranged protected below the outer housing layer and still be easily visible for a user.

In an alternate embodiment the transparent surface layer can also comprise the entire outer layer and/or the entire surface of the housing shell.

In an advantageous development of the inventive medical imaging apparatus there can be provision for the outer housing layer to comprise a transparent carrier layer and for the at least one sensor element and/or the at least one cleaning element to be arranged in the transparent carrier layer. The transparent carrier layer is may be formed from an amorphous material, in particular a non-crystalline material, such as for example SiO₂ Moreover the at least one cleaning element can likewise be embodied at least partly transparently, such as for example a heating element made of ITO and/or zinc aluminum oxide and/or carbon nanotubes etc. In this way a good visibility of components and/or elements of the layer structure that are arranged below the outer housing layer, such as for example an output element, can be achieved.

In an advantageous development of the inventive medical imaging apparatus there can be provision for the layer structure to have an insulation element, which is arranged between the at least one output element and the outer housing layer. In an exemplary embodiment, the insulation element comprises a thermal insulation element that protects the at least one output element from being affected by the heat from a heating element of the at least one cleaning element for example. Moreover, the insulation element can advantageously protect further components of the functional housing shell from the effect of heat from the heating element. The insulation element can be embodied in this case as an insulation layer, which is arranged in particular between the outer housing layer and the further housing layer with the at least one output element. Moreover, the insulation element can also be included in the carrier layer, wherein the carrier layer may simultaneously have a thermal insulating effect on the components and/or elements of the at least one functional housing layer arranged below it. This enables an advantageous protection, in particular a thermal protection of components and/or elements, in particular of the at least one output element, to be achieved during a cleaning by heating of a surface layer of the at least one functional housing shell.

In an advantageous development of the inventive medical imaging apparatus there can be provision for the layer structure to have a protective layer. The protective layer may be embodied electrically conductively, in order to guarantee a detection of a soiling and/or contamination by the sensor element arranged below the protective layer. Moreover, the protective layer is likewise embodied transparently. The protective layer in this case has a thickness of between 100 nm and 10 μm. Especially advantageously the protective layer can also have a thickness of between 200 nm and 800 nm. This advantageously enables the layers and/or components close to the surface, such as in particular the at least one sensor element and/or the at least one cleaning element, to be protected. In particular the components can be protected from an abrasive wiping movement during a cleaning. At the same time the protective layer for example allows a capacitive measurement for detecting contamination and/or soiling.

In an advantageous development of the inventive medical imaging apparatus there can be provision for the medical imaging apparatus to comprise a controller, which controls an automatic detection of a hygiene status on the at least one functional housing shell by means of the at least one sensor element and an automatic output of the hygiene status by means of the at least one output element.

The inventive controller comprises at least one computing module and/or a processor, wherein the controller is embodied for carrying out an automatic control of the at least one sensor element and of the at least one output element. Thus, in particular the controller is configured to execute computer-readable instructions for an automatic control of the at least one sensor element and of the at least one output element. In particular the controller comprises a memory, wherein computer-readable information is stored on the memory, wherein the controller is configured to load the computer-readable information from the memory and to execute the computer-readable information.

The components of the controller can for the most part be embodied in the form of software components. Basically, these components can also be realized in part, in particular when it is a matter of especially fast computations, in the form of software-supported hardware components, for example FPGAs or the like. Likewise, the interfaces needed, for example if it is only a matter of accepting data from other software components, can be embodied as software interfaces. They can however also be embodied as interfaces constructed from hardware, which are activated by suitable software. Naturally it is also conceivable for a number of the said components to be realized combined in the form of an individual software component or software-supported hardware component.

The controller can be integrated in this case within the at least one functional housing shell. Moreover, it is also conceivable for the controller to be arranged outside the at least one functional housing shell, for example when the controller is embodied for control of a number of functional housing shells. The controller may be configured to evaluate the detected sensor data.

This embodiment of the disclosure has the advantage that a rapid automatic detection and display of a hygiene status for a user can be provided. Advantageously the controller is embodied in such a way that contamination and/or a soiling during a medical imaging examination is stored and will only be indicated and/or displayed for the user after the medical imaging examination.

In an advantageous development of the inventive medical imaging apparatus there can be provision for the controller to control an automatic cleaning of the at least one functional housing shell, in particular of the surface of the functional housing shell, by means of the at least one cleaning element. For example, the controller can activate the at least one cleaning element in such a way that, with a soiling and/or contamination of the at least one functional housing shell, the cleaning begins as soon as the medical imaging examination is ended. In an alternate embodiment the cleaning can also only begin as soon as a user makes an entry that is detected as a trigger event for initiating a cleaning process of the controller.

In this way an automatic cleaning of the at least one functional housing shell can be achieved and thus the load on a cleaner can be relieved. Moreover, it can be ensured in this way that accidently forgetting a cleaning, for example because of time pressure on the cleaner, is prevented. Furthermore, a danger of infection for the patient can be reduced.

Furthermore, the disclosure is based on a method for cleaning a surface of a functional housing shell of a medical imaging apparatus, wherein the method comprises the following steps:
detection of a hygiene status of the surface of the functional housing shell by means of at least one sensor element,
output of the hygiene status to a user by means of at least one output element, and
provided a hygiene status is present that comprises a contamination and/or soiling of the surface, cleaning of the surface by means of the at least one cleaning element.

In an exemplary embodiment, the method is carried out with the aid of a controller of the functional housing shell, wherein the controller controls the individual units and/or components, in particular the at least one sensor element, the at least one output element and/or the at least one cleaning element for carrying out the method. In this way a hygiene status that comprises a contamination and/or soiling is not output by means of the at least one output element directly after the detection and/or determination of the hygiene status, but preferably only after the medical imaging examination has ended, in order to avoid disturbing the patient during the medical imaging examination. Moreover, the cleaning of the surface of the functional housing shell only begins after the medical imaging examination has ended. Moreover, the cleaning can also only be initiated after a user input.

What can advantageously be achieved by this is that a user, in particular a medical operator and/or a medical cleaner, is advantageously supported during a cleaning of the medical imaging apparatus after a medical imaging examination. In particular in this way a possible contamination of surfaces can be detected automatically. Visual display enables the user to detect a possible contamination and/or soiling especially quickly and effectively to estimate a possible cleaning effort and/or a cleaning workflow. Moreover, the user can also be given beneficial support during cleaning of the surfaces because of the at least one cleaning element. This also makes it possible to carry out a cleaning workflow in a time saving and efficient manner. Moreover, through the visual display, a danger of overlooking a surface during cleaning is reduced and thereby a danger of infection is also reduced for a patient.

The advantages of the inventive method for cleaning a surface of a functional housing shell of a medical imaging apparatus essentially correspond to the advantages of the inventive medical imaging apparatus, which have been set out in detail above. Features, advantages or alternate forms of embodiment can likewise be transferred to the other claimed subject matter and vice versa.

In an advantageous development of the inventive method there can be provision for there to be control of the sensor element, the output element and/or the cleaning element by means of a controller. This enables an advantageous automation of the method to be achieved and thus the load on a user during cleaning also to be relieved.

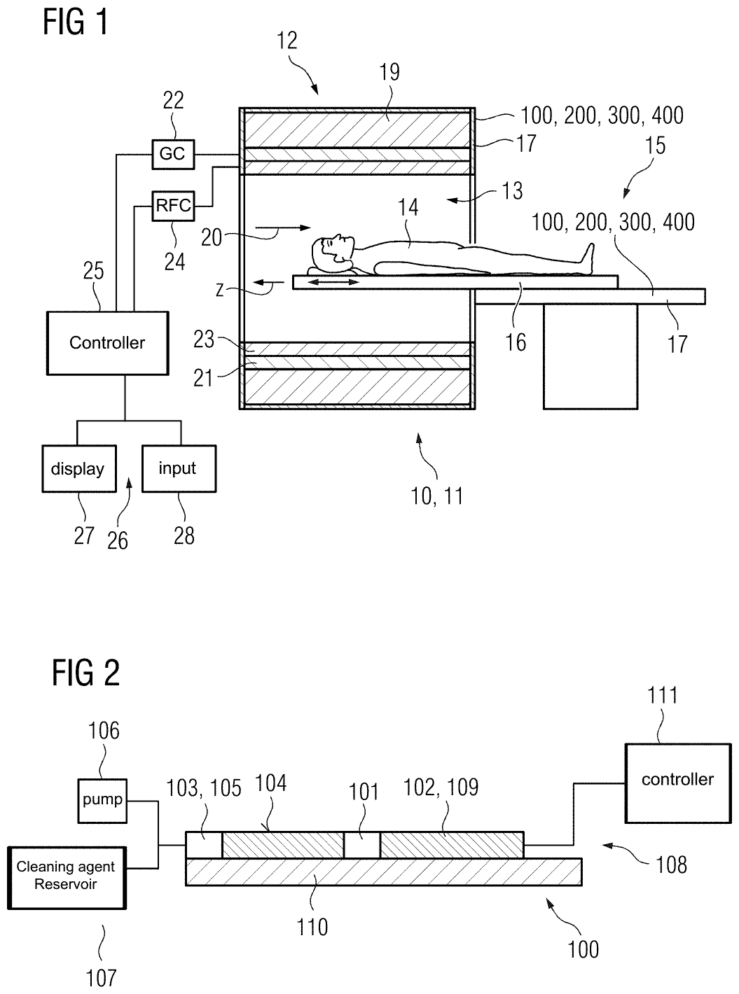

Shown schematically in FIG. 1 is a medical imaging apparatus 10. The medical imaging apparatus 10 in the present exemplary embodiment is formed by a magnetic resonance apparatus 11, wherein the present disclosure is explained by way of example with the aid of a magnetic resonance apparatus 11. The present disclosure is however not restricted to the embodiment of the medical imaging apparatus 10 as a magnetic resonance apparatus 11 and further embodiments of the medical imaging apparatus 10 are always conceivable.

The magnetic resonance apparatus 11 comprises a scanner 12 formed by a magnet unit. Moreover, the magnetic resonance apparatus 11 has a patient receiving area 13 for accommodating a patient 14. The patient receiving area 13 in the present exemplary embodiment is embodied cylindrical in shape and is surrounded in a circumferential direction by the scanner 12, in particular by the magnet unit, in a cylindrical shape. Basically, however an embodiment of the patient receiving area 13 that differs from this is always conceivable. The patient 14 can be pushed and/or moved by means of a patient support apparatus 15 of the magnetic resonance apparatus 11 into the patient receiving area 13. For this purpose, the patient support apparatus 15 has a patient table 16 embodied movably within the patient receiving area 13. In particular here the patient table 16 is supported movably in the direction of a longitudinal extent of the patient receiving area 13 and/or in the z direction.

Furthermore, the medical imaging apparatus 10, in particular the magnetic resonance apparatus 11, has a housing 17. The housing 17 is arranged in this case on the scanner 12 and also on the patient support apparatus 15. The housing 17 has at least one functional housing shell 100, 200, 300, 400. In the present exemplary embodiment, the housing 17 has a number of functional housing shells 100, 200, 300, 400, which may be arranged on areas of the scanner 12 and the patient support apparatus 13 that have a high probability of being contaminated and/or of being in contact with a patient 14 and/or with the medical operating personnel. In particular a front side of the scanner 12 and/or a housing arranged surrounding the patient receiving area 13 have functional housing shells 100, 200, 300, 400 here.

Moreover, the patient support apparatus 15 can also have functional housing shells 100, 200, 300, 400 in the area of the patient table 16.

The scanner 12, in particular the magnet unit, comprises a superconducting basic magnet 19 for creating a strong and in particular constant basic magnetic field 20. Furthermore, the scanner 12, in particular the magnet unit, has a gradient coil unit 21 for creation of magnetic field gradients, which are used for a spatial encoding during imaging. The gradient coil unit 21 is controlled by a gradient controller 22 of the magnetic resonance apparatus 11. Furthermore, the scanner 12, in particular the magnet unit, comprises a radio-frequency antenna unit 23 for excitation of a polarization that arises in the basic magnetic field 20 created by the basic magnet 19. The radio-frequency antenna unit 23 is controlled by a radio-frequency antenna controller 24 of the magnetic resonance apparatus 11 and radiates radio-frequency magnetic resonance sequences into the patient receiving area 13 of the magnetic resonance apparatus 11.

For control of the basic magnet 19, the gradient controller 22 and for control of the radio-frequency antenna controller 24 the magnetic resonance apparatus 11 has a system controller 25. The system controller 25 is configured to centrally control the magnetic resonance apparatus 11, such as, for example, the carrying out of a predetermined imaging gradient echo sequence. Moreover, the system controller 25 comprises an evaluation unit (e.g. processor), not shown in any greater detail, for evaluation of medical imaging data that is detected during the magnetic resonance examination. In an exemplary embodiment, the controller 25 includes processing circuitry configured to perform one or more operations and/or functions of the controller 25. One or more components of the controller 25 may include processing circuitry configured to perform one or more respective operations and/or functions of the component(s).

Furthermore, the medical imaging apparatus 10, in particular the magnetic resonance apparatus 11, comprises a user interface 26, which is connected to the system controller 25. Control information such as for example imaging parameters, and also reconstructed magnetic resonance images, can be shown on a display 27, on at least one monitor for example, of the user interface 26 for medical operating personnel. Furthermore, the user interface 26 has an input unit 28 (e.g. keyboard, mouse, touchscreen, microphone, etc.), by which information and/or parameters can be entered during a measurement process by the medical operating personnel.

The medical imaging apparatus 10 shown, in particular the magnetic resonance apparatus 11, can of course comprise further components that medical imaging apparatuses 10, in particular the magnetic resonance apparatuses 11, usually have. A general way in which a medical imaging apparatus 10, in particular the magnetic resonance apparatus 11, functions is moreover known to the person skilled in the art, so that a more detailed description of the further components will be dispensed with here.

The functional housing shells 100, 200, 300, 400 are described in greater detail below, wherein the description is given using a single housing shell 100, 200, 300, 400 as an example.

Shown in FIG. 2 is a first exemplary embodiment of a functional housing shell 100. The functional housing shell 100 has a sensor element 101, an output element 102 and a cleaning element 103. The functional housing shell 100 can in this case also have more than one sensor element 101 and/or more than one output element 102 and/or more than one cleaning element 103.

The sensor element 101 is embodied for detection of a hygiene status on the functional housing shell 100, in particular on the surface 104 of the functional housing shell 100. The sensor element 101 in this case can comprise a conductivity sensor, a moisture sensor, a temperature sensor, an optical sensor, a chemical sensor, an electrochemical sensor, a capacitive sensor, a resistive sensor and/or a gas sensor etc. The output element 102 is embodied for a visual output of the hygiene status and has an OLED, an LED, an electrochromic output element and/or an electroluminescent output element etc.

The cleaning element 103 is embodied for a cleaning of the surface 104 of the functional housing shell 100 and in the present exemplary embodiment comprises a channel structure with a fluid channel 105. A cleaning agent is conveyed in the fluid channel 105, which can emerge through an opening of the fluid channel 105 onto the surface 104 of the functional housing shell 100 and be distributed there. To this end the cleaning element 103 has a pump 106, for example a micro pump, and a cleaning agent reservoir 107, which are only shown schematically in FIG. 2. The pump 106 and the cleaning agent reservoir 107 can in this case also be comprised by the functional housing shell 100 or also centrally by the housing 17.

The functional housing shell 100 furthermore comprises a layer structure 108 with an outer housing layer 109. Arranged in this outer housing layer 109 is the sensor element 101, the output element 102 and the cleaning element 103. Moreover, the functional housing shell 100 comprises a further housing layer 110, which comprises a carrier layer. The functional housing shell 100 with the layer structure 108 has a thickness of between 50 μm and 5 mm in this case.

For a control of the sensor element 101, in particular for an evaluation of the sensor data detected and for determining the hygiene status, the housing 17 has a controller 111. The controller 111 can be comprised by the functional housing shell 100 in this case or also be embodied separately from this or also be comprised centrally by the housing 17. The controller 111 here is embodied, with the aid of the data of the sensor element 101 detected, to determine the hygiene status of the surface 104 of the functional housing shell 100. The hygiene status in this case can comprise a contamination and/or a soiling of the surface 104. Moreover, a hygiene status can also comprise information that no contamination and/or soiling of the surface 104 is present. Moreover, the hygiene status can also comprise a cleaning process. In an exemplary embodiment, the controller 111 includes processing circuitry configured to perform one or more operations and/or functions of the controller 111.

The output element 102 is also controlled by means of the controller 111. In an exemplary embodiment, the controller 111 is configured to control an automatic output of the hygiene status detected and/or determined by means of the at least one output element 102. For example, for a hygiene status that comprises a contamination and/or a soiling of the surface 104 of the functional housing shell 100, can be shown by output of a red light by means of the output element 102. If on the other hand a hygiene status is present that comprises no contamination and/or soiling of the surface 104 of the functional housing shell 100, this can be shown for example by output of a green light by means of the output element 102.

Moreover, a hygiene status that comprises a cleaning process of the surface 104 of the functional housing shell 100 can be shown by output of a blue light by means of the output element 102. In this case the output element 102 can be controlled by the controller 111 in such a way that a hygiene status is only displayed after the medical imaging examination, in order for example not to disturb a patient 14 during the medical imaging examination.

Moreover, the controller 11 is also configured to control a cleaning by means of the cleaning element 103. In an exemplary embodiment, the cleaning is tailored/controlled by the controller 111 to the medical imaging examination and is only undertaken here after the medical imaging examination. Moreover, it can also be that the controller 111 only starts a cleaning of the surface 104 of the functional housing shell 100 when an entry to start the cleaning process is made by a user. During a cleaning in this case a cleaning agent is pumped through the fluid channel 105 and can emerge at defined points on the surface 104 of the functional housing shell 100 and be distributed onto the surface 104.

For an activation of the sensor element 101, the output element 102 and the cleaning element 103, the controller 111 has corresponding control software and/or computer programs that, when executed in a processor of the controller 111, carry out the control of the sensor element 101, the output element 102 and the cleaning element 103.

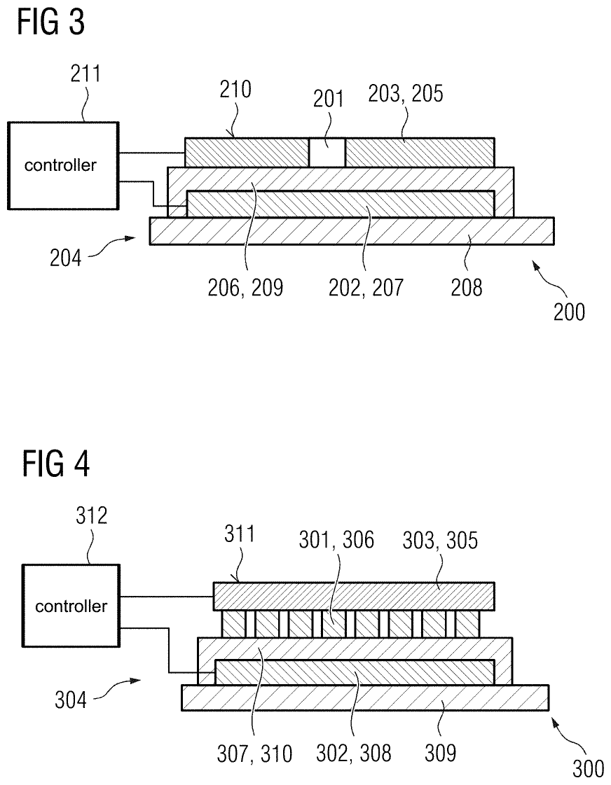

Shown in FIG. 3 is an alternate exemplary embodiment of the functional housing shell 200. Components, features and functions that essentially remain the same are basically labeled with the same reference characters. The following description essentially restricts itself to the differences from the exemplary embodiment in FIG. 2, wherein, as regards components, features and functions that remain the same, the reader is referred to the description of the exemplary embodiment in FIG. 2.

Shown in FIG. 3 is a second exemplary embodiment of the functional housing shell 200. The functional housing shell 200 here has a sensor element 201, an output element 202 and a cleaning element 203. The functional housing shell 200 can in this case also have more than one sensor element 201 and/or more than one output element 202 and/or more than one cleaning element 203. The sensor element 201 here is embodied as in the description for FIG. 2. Moreover, the output element 202 is also embodied as in the description for FIG. 2. The cleaning element 203 in the present exemplary embodiment comprises a heating element, such as for example a heating resistor and/or a heating element, which is inert on contact with air, such as in particular a copper wire and/or a heating element made of carbon.

The functional housing shell 200 here has a layer structure 204 with an outer housing layer 205. The outer housing layer 205 here comprises a surface layer of the functional housing shell 200. Arranged in this outer housing layer 205 is the cleaning element 203 and the sensor element 201. Moreover, the layer structure 204 of the functional housing shell 200 has further housing layers 206, 207, 208, wherein one of the further housing layers 207 comprises the output element 202. Arranged between the outer housing layer 204 and the housing layer 207 comprising the output element 202 is a housing layer 206 with an insulation element 209 and/or an insulation layer, in order to protect the output element 202 against overheating. In an exemplary embodiment, the insulation element 209 and/or the insulation layer is embodied thermally insulating.

In order to make an output of the output element 202 visible for a user, the insulation element 209 and the outer housing layer 205 is embodied at least partly transparently. To this end for example the housing layer 206 with the insulation element 209 and/or the insulation layer is made of glass and/or a transparent plastic, in particular heat-resistant transparent plastic, such as for example a polyimide. For this purpose, for example the heating element, in particular the heating resistor, has a structure made of indium tin oxide (ITO) in the outer housing layer 205, wherein the structure made of ITO can comprise transparent conductor tracks for a heatable layer, for example. For example, the outer housing layer 205 and/or a surface 210 of the functional housing shell 200 here is heated up to appr. 200° C. In an exemplary embodiment, the heating up can take place within a few seconds by targeted heat pulses. Moreover, the outer housing layer 205 can also comprise a carrier structure and/or a carrier material, in which the heating element is arranged, wherein the carrier structure and/or the carrier material may be transparent, such as for example from silicon dioxide (SiO₂).

Moreover, the layer structure 204 of the functional housing shell 200 comprises a further housing layer 208, which comprises a carrier layer. In an exemplary embodiment, the carrier layer is the housing layer 208 of the layer structure 204 furthest from the surface.

The functional housing shell 200 furthermore likewise has a controller 211, wherein a way in which the controller 211 functions corresponds to the descriptions given for FIG. 2. In particular the mode of operation and/or activation of the sensor element 201 and of the output element 202 is as described for FIG. 2. The activation of the cleaning element 203 is also in accordance with the description for FIG. 2, wherein in the present exemplary embodiment, when the cleaning element 203 is activated by the controller 211, the surface 210 of the functional housing shell 200 is briefly heated up to appr. 200° C. In an exemplary embodiment, the controller 211 includes processing circuitry configured to perform one or more operations and/or functions of the controller 211.

Shown in FIG. 4 is an alternate exemplary embodiment of the functional housing shell 200. Components, features and functions that essentially remain the same are basically labeled with the same reference characters. The following description essentially restricts itself to the differences from the exemplary embodiments in FIGS. 2 and 3, wherein, as regards components, features and functions that remain the same, the reader is referred to the description of the exemplary embodiments in FIGS. 2 and 3.

Shown in FIG. 4 is a third exemplary embodiment of the functional housing shell 300. The functional housing shell 300 here has a sensor element 301, an output element 302 and a cleaning element 303. The functional housing shell 300 can in this case also have more than one sensor element 301 and/or more than one output element 302 and/or more than one cleaning element 303.

Here too the functional housing shell 300 has a layer structure 304 with an outer housing layer 305 and further housing layers 306, 307, 308 309. In this exemplary embodiment, the outer housing layer 305 exclusively comprises the cleaning element 303, which is embodied according to the description given for FIG. 3. Moreover, one of the further housing layers 307 has an insulation element 310 and/or an insulation layer, which is embodied according to the description given for FIG. 3. A further housing layer 308 comprises the output element 302, as has already been described in the description given for FIG. 3. Moreover, the layer structure 304 of the functional housing shell 300 comprises a further housing layer 309, which comprises a carrier layer. In an exemplary embodiment, the carrier layer is the housing layer 309 of layer structure 304 furthest from the surface.

Arranged between the outer housing layer 305 and the further housing layer 307 with the insulation element 310 and/or the insulation layer is a further housing layer 306 of the functional housing shell 300, which comprises the sensor element 301. In the present exemplary embodiment, the sensor element 301 comprises a tactile sensor, such as for example a capacitive sensor for detection of a contact between a patient 14 and/or the user and a surface 311 of the functional housing shell 300.

The functional housing shell 300 furthermore likewise has a controller 312, wherein the way in which the controller functions corresponds to the description given for FIG. 2. In particular a mode of operation and/or activation of the sensor element 301, of the output element 302 and of the cleaning element 303 is as described for FIGS. 2 and 3. In an exemplary embodiment, the controller 312 includes processing circuitry configured to perform one or more operations and/or functions of the controller 312.

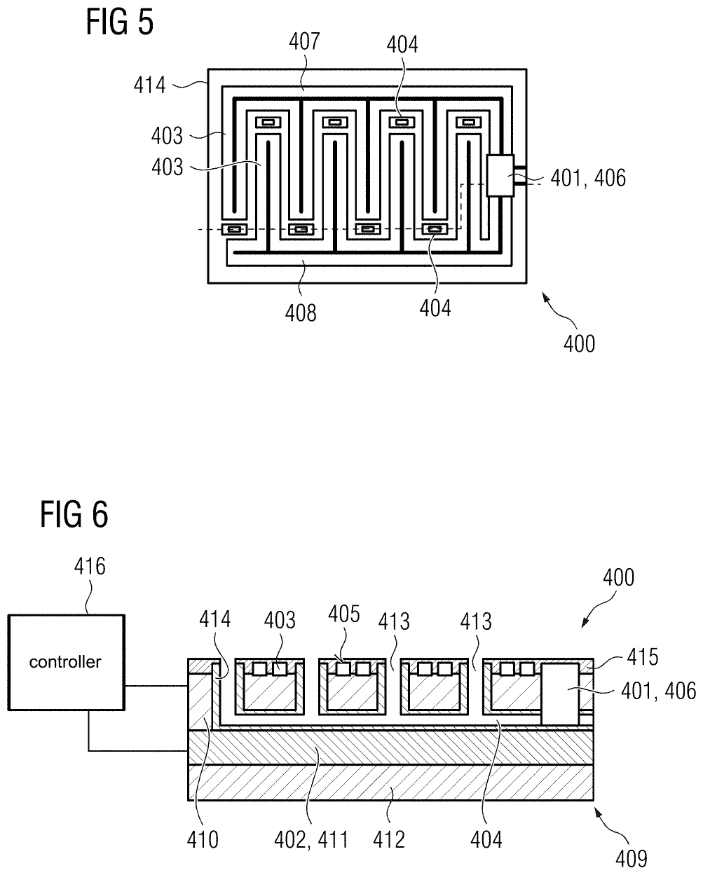

Shown in FIGS. 5 and 6 is an alternate exemplary embodiment of the functional housing shell 400. Components, features and functions that essentially remain the same are basically labeled with the same reference characters. The following description essentially restricts itself to the differences from the exemplary embodiments in FIG. 2 to FIG. 4, wherein, as regards components, features and functions that remain the same, the reader is referred to the description of the exemplary embodiments in FIG. 2 to FIG. 4.

Shown in FIGS. 5 and 6 is a fourth exemplary embodiment of the functional housing shell 400. The functional housing shell 400 here has a sensor element 401, an output element 402 and two cleaning elements 403, 404. The functional housing shell 400 in this case can also have more than one sensor element 401 and/or more than one output element 402. A first cleaning element 403 comprises a heating element with two heating circuits 407, 408. By means of the two heating circuits 407, 408 the surface 405 of the functional housing shell 400 can be briefly heated up, for example to appr. 200° C. The first cleaning element 403 in the present exemplary embodiment moreover has a control element (controller) 406 that connects the two heating circuits 407, 408 to one another. Moreover, by means of the control element 406, the heating circuits 407, 408 can be capacitively read out and thus the first cleaning element 403, in particular the two heating circuits 407, 408 together with the control element 406, can function as a sensor element 401 of the functional housing shell 400. In this way contacts between a person and the surface 405 of the functional housing shell 400 and/or soiling, by fluids for example, can be detected. A second cleaning element 404 comprises a fluid channel and is embodied analogously to the cleaning element 203 in the way in which it operates, in particular the fluid channel, in FIG. 2. In this case the second cleaning element 404 likewise has a pump not shown in any greater detail and a cleaning reservoir not shown in any greater detail. In an exemplary embodiment, the controller 406 includes processing circuitry configured to perform one or more operations and/or functions of the controller 406.

Here too the functional housing shell 400 has a layer structure 409 with an outer housing layer 410 and further housing layers 411, 412. Arranged in the outer housing layer 410 are the two cleaning elements 403, 404 and thus also the sensor element 401. As can be seen in FIGS. 5 and 6, regular openings 413 are arranged in areas between the two heating circuits 407, 408, through which a cleaning fluid can be distributed onto the surface 405 of the functional housing shell 400. Moreover, the outer housing layer 410 comprises a transparent carrier layer 414, wherein the two cleaning elements 403, 404 and thus also the sensor element 401 are arranged within the carrier layer 414. In an exemplary embodiment, the two cleaning elements 403, 404 are also arranged transparently for this purpose. A side of the carrier layer 414 facing towards a further housing layer 411 serves at the same time as a thermal insulation element for a further housing layer 411 arranged below the outer housing layer 410. The output element 402, which is embodied according to the description given for FIG. 2, is arranged in this further housing layer 411.

Furthermore, the functional housing shell 400, in particular the layer structure 409 of the functional housing shell 400, has a protective layer 415, which is arranged on the surface 405 of the functional housing shell 400. The protective layer 415 has the task of protecting the surface 405 of the functional housing shell 400, for example from abrasive wiping movements during a cleaning of the surface 405 of the functional housing shell 400. The protective layer 415 here has a thickness of between 100 nm and 10 μm. In an exemplary embodiment, the protective layer 415 can also have a thickness of between 200 nm and 800 nm. The protective layer 415 may be embodied electrically conductively, in order to guarantee detection of a soiling and/or contamination by the sensor element 401 arranged below the protective layer 415.

Moreover, the layer structure 409 of the functional housing shell 400 comprises a further housing layer 412, which comprises a carrier layer. In an exemplary embodiment, the carrier layer is the housing layer 412 of the layer structure 409 furthest away. The output element 402 in this case is arranged between the carrier layer and the outer housing layer 410.

The functional housing shell 400 furthermore has a controller 416, wherein the sensor element 401, the output element 402 and the cleaning elements 403, 404 are activated according to the description given in FIGS. 2 and 3. In an exemplary embodiment, the controller 416 includes processing circuitry configured to perform one or more operations and/or functions of the controller 416.

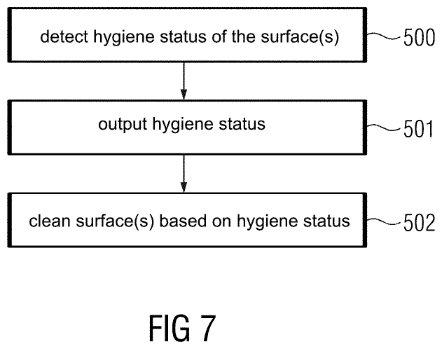

Shown in FIG. 7 is a method for cleaning a surface 104, 210, 311, 405 of a functional housing shell 100, 200, 300, 400 of a medical imaging apparatus 10. In a first method step 500 a hygiene status of the surface 104, 210, 311, 405 of the functional housing shell 100, 200, 300, 400 is detected by means of at least one sensor element 101, 201, 301, 401 of the functional housing shell 100, 200, 300, 400. Here the data detected by the sensor element 101, 201, 301, 401 is evaluated by the controller 111, 211, 312, 416 in respect of a hygiene status. The detection by means of the sensor element 101, 201, 301, 401 is already undertaken in this case during a preparation of the patient 14 for the medical imaging examination. Moreover, the detection is also undertaken during the medical imaging examination.

In a further method step 501 the hygiene status is output to a user by means of an output element 102, 202, 302, 402 of the functional housing shell 100, 200, 300, 400. The hygiene status is output in this case under the control of the controller 111, 211, 312, 416. In an exemplary embodiment, the status is output by means of the output element 102, 202, 302, 402 after the medical imaging examination has ended. For example, an illumination of a red light by the output element 102, 202, 302, 402 can signal to the user that a contamination and/or soiling of the surface 104, 210, 311, 405 of the functional housing shell 100, 200, 300, 400 is present. On the other hand, an illumination of a green light by the output element 102, 202, 302, 402 can signal to the user that the surface 104, 210, 311, 405 of the functional housing shell 100, 200, 300, 400 is free from a contamination and/or soiling. Furthermore, it is also conceivable for an illumination of a blue light by the output element 102, 202, 302, 402 to signal to the user that a cleaning process is ongoing.

Provided a hygiene status is present that comprises a contamination and/or soiling of the surface 104, 210, 311, 405 of the functional housing shell 100, 200, 300, 400, the surface 104, 210, 311, 405 is cleaned by means of at least one cleaning element 103, 203, 303, 403, 404 of the functional housing shell 100, 200, 300, 400. The cleaning of the surface is in this case controlled by means of the controller 111, 211, 312, 416. In an exemplary embodiment, the cleaning takes place after the medical imaging examination has ended. Moreover, it can be that a start entry has to be made by means of the input unit of the medical imaging apparatus before a cleaning process is begun.

Although the disclosure has been illustrated and described in greater detail by the exemplary embodiments, the disclosure is not restricted by the disclosed examples and other variations can be derived herefrom by the person skilled in the art, without departing from the scope of protection of the disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A medical imaging apparatus comprising:
a scanner;
a patient support apparatus; and
a housing, which is arranged on the scanner and/or on the patient support apparatus, the housing including a functional housing shell, wherein the functional housing shell includes:
at least one sensor configured to detect a hygiene status on the functional housing shell;
interface configured to visually display the hygiene status; and
at least one cleaning element configured to clean a surface of the functional housing shell.

2. The medical imaging apparatus as claimed in claim 1, wherein the at least one sensor element comprises a conductivity sensor, a moisture sensor, a temperature sensor, an optical sensor, a chemical and/or electrochemical sensor, a capacitive sensor, a resistive sensor, and/or gas sensor.

3. The medical imaging apparatus as claimed in claim 1, wherein the interface comprises an organic light-emitting diode (OLED), a light-emitting diode (LED), an electrochromic output element, and/or an electroluminescent output element.

4. The medical imaging apparatus as claimed in claim 1, wherein the at least one cleaning element comprises a heating element and/or a fluid channel.

5. The medical imaging apparatus as claimed in claim 1, wherein the functional housing shell has a layer structure with an outer housing layer, the at least one cleaning element and/or the at least one sensor element being arranged in the outer housing layer.

6. The medical imaging apparatus as claimed in claim 5, wherein the layer structure has at least one further housing layer, in which the interface is arranged.

7. The medical imaging apparatus as claimed in claim 5, wherein the outer housing layer is at least partly transparent.

8. The medical imaging apparatus as claimed in claim 5, wherein the outer housing layer comprises a transparent carrier layer and the at least one sensor element and/or the at least one cleaning element is arranged in the transparent carrier layer.

9. The medical imaging apparatus as claimed in claim 5, wherein the layer structure has an insulator arranged between the interface and the outer housing layer.

10. The medical imaging apparatus as claimed in claim 5, wherein the layer structure has a protective layer.

11. The medical imaging apparatus as claimed in claim 1, further comprising a controller configured to:
control the at least one sensor to automatically detect the hygiene status on the functional housing shell; and
control the interface to automatically output the hygiene status.

12. The medical imaging apparatus as claimed in claim 11, wherein the controller is further configured to control the at least one cleaning element to automatically clean the functional housing shell.

13. A method for cleaning a surface of a functional housing shell of a medical imaging apparatus, the method comprising:
detecting, by a sensor, a hygiene status of the surface of the functional housing shell;
outputting, by an output interface, the hygiene status to a user;
determining, by a controller and based on the hygiene status, whether the surface is contaminated and/or soiled; and
controlling, by the controller and based on whether the surface is contaminated and/or soiled, a cleaning element to clean the surface.

14. The method as claimed in claim 13, wherein detecting the hygiene status includes controlling, by the controller, the sensor to analyze the surface to detect the hygiene status element.

15. The method as claimed in claim 13, wherein controlling the cleaning element comprises applying a cleaning agent and/or a disinfection agent onto the surface.

16. The method as claimed in claim 13, wherein controlling the cleaning element comprises irradiating, by a radiation source of the cleaning element, the surface.

17. The method as claimed in claim 13, wherein controlling the cleaning element comprises heating, by a heater of the cleaning element, the surface.

18. A non-transitory computer-readable storage medium with an executable program stored thereon, that when executed, instructs a processor to perform the method of claim 13.
